(19) United States
(12) Patent Application Publication
(10) Pub. No.: US 2025/0339358 A1
(43) Pub. Date: Nov. 6, 2025

(54) NOVEL USE OF A BLEND OF SUGARS COMPRISING PSICOSE, MANNOSE, FRUCTOSE AND GLUCOSE

(71) Applicant: DSM IP ASSETS B.V., Maastricht (NL)

(72) Inventors: Dominik IMFELD, Kaiseraugst (CH); Volker ROSENBERGER, Kaiseraugst (CH)

(21) Appl. No.: 18/862,342

(22) PCT Filed: May 11, 2023
(86) PCT No.: PCT/EP2023/062550

§ 371 Date: Nov. 1, 2024

(30) Foreign Application Priority Data
May 11, 2022 (EP) 22172777.9

(51) Int. Cl. A61K 8/60; A61Q 19/00

(52) U.S. Cl. CPC A61K 8/60; A61Q 19/00; A61K 2800/5922

(57) ABSTRACT

The present invention relates to the use of the use of a blend of sugars comprising psicose, mannose, fructose and glucose to increase the expression of cytokeratin-1 in skin to strengthen skin's self-defense mechanisms and maintain skin integrity.

The present invention relates to the use of the use of a blend of sugars comprising psicose, mannose, fructose and glucose to increase the expression of cytokeratin-1 in skin to strengthen skin's self-defense mechanisms and maintain skin integrity.

Keratins are keratin proteins found in the intracytoplasmic cytoskeleton of epithelial tissue. They are an important component of intermediate filaments, which help cells resist mechanical stress.

Cytokeratin-1 is a major constituent of the intermediate filament cytoskeleton in suprabasal epidermis. Reduced levels of cytokeratin-1 may cause epidermolytic ichthyosis in humans. In search of the largely unknown pathomechanisms and the role of keratins in barrier formation and inflammation control, cytokeratin-1 has been found to be crucial for maintenance of skin integrity and participates in an inflammatory network in murine keratinocytes.

Thus, there is an ongoing need for ingredients that are able to stimulate the expression of cytokeratin-1 in skin tissue, in particular in the presence of microorganisms such as in particular in skin colonized by one or more of C. acnes, S. Epidermidis, C. striatum and S. aureus

Without wishing to be bound by any specific theory or mechanism of action, the promotion of skin integrity may thus be due to the presence of cytokeratin-1, known to be essential for the proper differentiation of simple and stratified epithelial tissues. The blend of sugars of the present invention can thus be used to strengthen skin's self-defense mechanisms and maintain skin integrity.

Thus, in a first embodiment, the present invention relates to a composition comprising an effective amount of a blend of sugars comprising psicose, mannose, fructose and glucose for its use in a dermatological treatment as an agent for increasing the expression of cytokeratin-1 in skin tissue, in particular in cases where the skin is or is endangered to be colonized with pathogenic bacteria such as in particular S. aureus. Said use is particular suitable to strengthen skin's self-defense mechanisms and maintain skin integrity, in particular in the presence of pathogenic bacteria such as S. aureus. Said use is thus able to mitigate and/or counteract negative ailments associated with a colonialization of the skin with pathogenic bacteria such as in particular with S. aureus.

In a further embodiment, the present invention relates to a method to prevent or treat skin conditions caused by a reduced expression of cytokeratin-1 in skin tissue, in particular in skin colonized with pathogenic bacteria, preferably with S. aureus, said method comprising the steps of:
a) providing a cosmetic or dermatological composition comprising a blend of sugars comprising psicose, mannose, fructose and glucose, and
b) applying an amount of said composition to human skin that is sufficient to increase the expression of cytokeratin-1, preferably to skin colonized with S. aureus or endangered to be exposed to S. aureus.

The expression of cytokeratin-1 according to the present invention is understood to strengthen skin's self-defense mechanisms and maintain skin in a healthy stage.

In a further aspect, the present invention relates to a blend of sugars comprising psicose, mannose, fructose and glucose (and/or cosmetic or dermatological compositions comprising said blend of sugar) for: the use in the prevention and/or treatment of a skin condition associated with a reduced cytokeratin-1 expression; in the treatment of a skin condition in which a reduced cytokeratin-1 expression contributes to the pathology, and/or symptoms, and/or progression of said skin condition, in increasing the expression of cytokeratin-1 in skin tissue—in particular in skin colonized with, exposed to or endangered to be exposed to S. aureus (including in a subject in need thereof), and/or as a cytokeratin-1 expression promotor.

In another aspect, there is provided a use of a blend of sugars comprising psicose, mannose, fructose and glucose (and/or cosmetic or dermatological compositions comprising said blend of sugars): in the treatment of a disease or disorder associated with a reduced cytokeratin-1 expression; in the treatment of a disease or disorder in which a reduced cytokeratin-1 expression contributes to the pathology, and/or symptoms, and/or progression of said disease/disorder; in expressing cytokeratin-1 in skin tissue—in particular in skin colonized with, exposed to or endangered to be exposed to S. aureus, and/or as a cytokeratin-1 expression promotor (including in a subject in need thereof).

In another aspect, there is provided a use of a blend of sugars comprising psicose, mannose, fructose and glucose (and/or cosmetic or dermatological compositions comprising said blend of sugars) in the manufacture of a medicament for: the treatment of a disease or disorder associated with a reduced expression of cytokeratin-1; the treatment of a disease or disorder in which a reduced cytokeratin-1 expression contributes to the pathology, and/or symptoms, and/or progression, of said disease/disorder, and/or expression of cytokeratin-1—in particular in skin colonized with, exposed to or endangered to be exposed to S. aureus (including in a subject in need thereof).

In another aspect, there is provided a method of treating a disease or disorder in which a reduced cytokeratin-1 expression contributes to the pathology, and/or symptoms, and/or progression, of said disease/disorder, comprising administering an effective amount of a blend of sugars comprising psicose, mannose, fructose and glucose, for instance to a subject (in need thereof), preferably to a subject, where the skin is colonized with, exposed to or endangered to be exposed to S. aureus.

The present invention also relates to a blend of sugars comprising psicose, mannose, fructose and glucose as cytokeratin-1 expression promotor, in particular in skin tissue.

The term ‘skin’ as used in this document, is meant to include the external surface of mammals, especially humans and includes the skin and the scalp. Preferred skin in all embodiments of the present invention is facial and body skin such as most preferably facial skin.

The term ‘prevention’ as used herein refers to lessening the risk of developing a skin condition associated with a reduced cytokeratin-1 expression.

The term ‘treatment’ as used herein refers to an amelioration of symptoms, delaying the onset and/or reduction of the duration of any diseases associated with a reduced cytokeratin-1 expression. The treatment can be prophylactic (cosmetic) or therapeutic. Preferably, the treatment is prophylactic.

The term ‘cytokeratin-1 expression promotor’ as used herein refers to a compound capable of stimulating the expression of cytokeratin-1 in skin tissue. The cytokeratin-1 expression in skin tissue may be reduced by the presence of pathogenic bacteria present on the skin such as e.g. the presence of S. aureus.

In particular advantageous embodiments according to the present invention, the uses and methods according to the present invention take place on human skin, wherein the microbiome of said skin comprises one or more, preferably all of Cutibacterium acnes (C. acnes), S. Epidermidis, Corynebacterium striatum (C. striatum) and Staphylococcus aureus (S. aureus). The uses and methods can also take place on human skin wherein the microbiome of said skin comprises one or more, preferably all of C. acnes, S. Epidermidis and C. striatum and wherein the human skin is exposed to or endangered to be exposed to S. aureus.

The term ‘effective amount’ as used herein refers to an amount necessary to obtain the physiological effect. The physiological effect may be achieved by one application dose or by repeated applications. The dosage administered may, of course, vary depending upon known factors, such as the physiological characteristics of the particular cosmetic or dermatological composition comprising a blend of sugars comprising psicose, mannose, fructose and glucose and its mode and route of administration; the age, the nature and extent of the symptoms; the kind of concurrent treatment; the frequency of treatment; and the effect desired and can be adjusted by a person skilled in the art.

Preferably, the use level of psicose in all embodiments of the present invention is selected in the range from 0.0001 to 0.5 wt.-%, more preferably in the range from 0.0005 to 0.25 wt.-%, most preferably in the range from 0.00075 to 0.2 wt.-% such as in the range from 0.001 to 0.15 wt.-%, based on the total weight of the composition. Further suitable ranges encompass 0.0001 to 0.1 wt.-%; 0.0005 to 0.1 wt.-%, 0.00075 to 0.1 wt.-%, 0.00075 to 0.1 wt.-%, 0.001 to 0.1 wt.-%, 0.005 to 0.1 wt.-%, 0.01 to 0.1 wt.-%, and 0.01 to 0.1 wt.-%, 0.001 to 0.05 wt.-%, and 0.01 to 0.05 wt.-%.

Preferably, the use level of mannose in all embodiments of the present invention is selected in the range from 0.0001 to 0.5 wt.-%, more preferably in the range from 0.0005 to 0.25 wt.-%, most preferably in the range from 0.00075 to 0.2 wt.-% such as in the range from 0.001 to 0.15 wt.-%, based on the total weight of the composition. Further suitable ranges encompass 0.0001 to 0.1 wt.-%; 0.0005 to 0.1 wt.-%, 0.00075 to 0.1 wt.-%, 0.00075 to 0.1 wt.-%, 0.001 to 0.1 wt.-%, 0.005 to 0.1 wt.-%, 0.01 to 0.1 wt.-%, and 0.01 to 0.1 wt.-%, 0.001 to 0.05 wt.-%, and 0.01 to 0.05 wt.-%.

Preferably, the use level of fructose in all embodiments of the present invention is selected in the range from 0.01 to 2 wt.-%, more preferably in the range from 0.05 to 1 wt.-%, most preferably in the range from 0.05 to 0.75 wt.-% such as in the range from 0.05 to 0.5 wt.-%, based on the total weight of the composition. Further suitable ranges encompass 0.01 to 1 wt.-%; 0.05 to 1 wt.-%, 0.01 to 0.5 wt.-%; 0.05 to 0.5 wt.-%, 0.1 to 1 wt.-%; 0.1 to 0.5 wt.-%.

Preferably, the use level of glucose in all embodiments of the present invention is selected in the range from 0.01 to 3 wt.-%, more preferably in the range from 0.05 to 2.5 wt.-%, most preferably in the range from 0.075 to 2 wt.-% such as in the range from 0.1 to 1 wt.-%, based on the total weight of the composition. Further suitable ranges encompass 0.01 to 1 wt.-%; 0.05 to 1 wt.-%, 0.01 to 0.5 wt.-%; 0.05 to 0.5 wt.-%, 0.1 to 1 wt.-%; 0.1 to 0.5 wt.-%.

In all embodiments of the present invention all isomers of the sugars can be used, i.e. the respective D-and L-isomers, as well as mixtures thereof. The natural ones, i.e. the D-isomers however, being particularly preferred in all embodiments.

Preferably, in all embodiments of the present invention the sugars are incorporated into the composition according to the present invention in the form of a, preferably aqueous, sugar premix A comprising
a) from 1 to 5 wt.-%, based on the sugar premix, of psicose,
b) from 1 to 5 wt.-%, based on the sugar premix, of mannose,
c) from 10 to 30 wt.-%, based on the sugar premix, of fructose, and
d) from 15 to 60 wt.-%, based on the sugar premix, of glucose.

More preferably, in all embodiments of the present invention the sugar premix is an aqueous sugar premix B comprising
a) from 1 to 5 wt.-%, based on the sugar premix, of psicose,
b) from 1 to 5 wt.-%, based on the sugar premix, of mannose,
c) from 10 to 30 wt.-%, based on the sugar premix, of fructose, and
d) from 15 to 35 wt.-%, based on the sugar premix, of glucose.

Most preferably, in all embodiments of the present invention the aqueous sugar premix is an aqueous sugar premix C comprising
a) from 2 to 3 wt.-%, based on the sugar premix, of psicose,
b) from 1.5 to 3 wt.-%, based on the sugar premix, of mannose,
c) from 10 to 20 wt.-%, based on the sugar premix, of fructose, and
d) from 20 to 30 wt.-%, based on the sugar premix, of glucose.

The term ‘sugar premix’ as used herein refers to a pre-blended mixture comprising psicose, mannose, fructose and glucose in the amounts indicated herein. Said sugar premix can either be prepared by admixing the individual sugars or by isomerization of glucose, preferably of plant derived glucose, before incorporation into the compositions according to the present invention. The aqueous sugar premix preferably comprises from 25 to 50 wt.-% of water, based on the total aqueous premix of water.

Isomerization of glucose is well known to a person skilled in the art. Preferably, the isomerization process comprises (a) dissolving glucose in water followed by (b) isomerization said glucose in the presence of a base, preferably in the presence of sodium hydroxide, more preferably at a temperature selected in the range from 25 to 100° C. and (c) purifying the resulting reaction mixture by chromatography and optionally filtration.

In particular when the sugar premix is prepared by isomerization of glucose, the sugar premix may further comprise up to 7.5 wt.-%, preferably up to 5 wt.-% of further sugars selected from the group of pentoses, hexoses, di-and oligosaccharides, such as in particular galactose, sorbose as well as di-and oligosaccharides. Preferably, the amount of galactose and/or sorbose in the sugar premix according to the present invention is selected in the range from 0 to 4 wt.-%, such as in the range from 1 to 3 wt.-%. The residual amounts of sugars comprised in the premix are di-and oligosaccharides.

In a particular embodiment the sugar premix according to the present invention further comprises (e) sorbose in amounts selected in the range from1 to 5 wt.-%, preferably in the range from 1 to 3 wt.-%, based on the sugar premix.

Preferably, the use level of sorbose in all embodiments of the present invention is selected in the range from 0.0001 to 0.5 wt.-%, more preferably in the range from 0.0005 to 0.25 wt.-%, most preferably in the range from 0.00075 to 0.2 wt.-% such as in the range from 0.001 to 0.15 wt.-%, based on the total weight of the composition. Further suitable ranges encompass 0.0001 to 0.1 wt.-%; 0.0005 to 0.1 wt.-%, 0.00075 to 0.1 wt.-%, 0.00075 to 0.1 wt.-%, 0.001 to 0.1 wt.-%, 0.005 to 0.1 wt.-%, 0.01 to 0.1 wt.-%, and 0.01 to 0.1 wt.-%, 0.001 to 0.05 wt.-%, and 0.01 to 0.05 wt.-%.

In a particular advantageous embodiment, the sugar premix according to the present invention is an aqueous sugar premix, i.e. wherein the sugars are dissolved in water.

A particularly suitable aqueous sugar premix according to the present invention (sugar premix D) consists essentially of
a) from 1 to 5 wt.-%, preferably from 2 to 3 wt.-%, based on the aqueous sugar premix, of psicose,
b) from 1 to 5 wt.-%, preferably from 1.5 to 3 wt.-%, based on the aqueous sugar premix, of mannose,
c) from 10 to 30 wt.-%, preferably from 10 to 20 wt.-%, based on the aqueous sugar premix, of fructose,
d) from 15 to 30 wt.-%, preferably from 20 to 30 wt.-%, based on the aqueous sugar premix, of glucose, and optionally
e) up 7.5 wt.-%, preferably up to 5 wt.-%, based on the aqueous sugar premix, of further sugars,
f) 0.1 to 2 wt.-%, based on the aqueous sugar premix, of an additive, preferably citric acid and/or a salt thereof such as preferably the sodium salt, and
g) up to 100%, based on the aqueous sugar premix, of water.

The term consisting essentially of as used herein means that the total amount of the ingredients a) to g) ideally sums up to 100 wt.-%. It is however not excluded that small amount of unknown (sugar) impurities, e.g. derived from the isomerization process of glucose may be present.

An aqueous sugar premix according to the present invention is e.g. commercially available as Pentavitin® at DSM Nutritional Products Ltd.

The total amount of sugar premix to be incorporated into the compositions according to the present invention is preferably selected in the range from 0.01 to 10 wt.-%, more preferably in the range from 0.1 to 7.5 wt.-%, most preferably in the range from 0.2 to 5 wt.-%, based on the total weight of the aqueous composition. Further suitable ranges are from 0.25 to 2.5 wt.-% and from 0.5 to 2 wt.-%. Particularly preferred ranges according to the present invention are from 0.2 to 1 wt.-%, more preferably from 0.25 to 0.75 wt.-%, such as from 0.3 to 0.6 wt.-%.

The term ‘dermatological’ as used herein may refer to cosmetic (non-therapeutic) as well as pharmaceutical (therapeutic) treatments. In all embodiments of the present invention cosmetic treatments i.e. treatments intended for beautifying the skin are preferred.

The term ‘cosmetic or dermatological composition’ as used herein refers to compositions, which are used to treat, care for or improve the appearance of the skin and/or the scalp. Particular advantageous cosmetic or dermatological compositions are skin care preparations.

In all embodiments of the present invention preferably the cells are skin cells such as in particular epithelial cells, especially keratinocytes, melanocytes, fibroblasts or dendritic cells.

In all embodiments of the present invention preferably the treatment is non-therapeutic, i.e. cosmetic.

Preferably, the amount of the cosmetic or dermatological composition according to the present invention to be applied to the skin is selected in the range of 0.1 to 3 mg/cm² skin, such as preferably in the range of 0.1 to 2 mg/cm² skin and most preferably in the range of 0.5 to 2 mg/cm² skin.

In all embodiments of the present invention, preferably, the composition is applied on the skin before or after exposure or contact with pathogenic bacteria such as in particular S. aureus, preferably before.

The cosmetic or dermatological compositions according to the invention are intended for topical application, which is to be understood as the external application to keratinous substances, such as in particular the skin.

As the cosmetic or dermatological compositions according to the invention are intended for topical application, they comprise a physiologically acceptable medium, that is to say a medium compatible with keratinous substances, such as in particular the skin. In particular the physiologically acceptable medium is a cosmetically, respectably dermatologically acceptable carrier.

The term ‘cosmetically acceptable carrier’ respectively ‘dermatologically acceptable carrier’ as used herein refers to a physiologically acceptable medium which is compatible with keratinous substances. Suitable carriers are well known in the art and are selected based on the end-use application. Preferably, the carriers of the present invention are suitable for application to skin (e.g., sunscreens, creams, milks, lotions, masks, serums, hydrodispersions, foundations, creams, creamgels, or gels etc.). Such carriers are well-known to one of ordinary skill in the art and can include one or more compatible liquid or solid filler diluent, excipient, additive or vehicle which are suitable for application to skin. The exact amount of carrier will depend upon the level of the active(s), respectively active blends, and any other optional ingredients that one of ordinary skill in the art would classify as distinct from the carrier (e.g., other active components). The cosmetic compositions of the present invention preferably comprise from about 70% to about 99.999%, more preferably from about 85% to about 99.99%, still more preferably from 90% to about 99%, and most preferably, from about 93% to about 98%, by weight of the cosmetic composition, of a carrier.

The cosmetic compositions of the present invention can be formulated into a wide variety of product types, including creams, waxes, pastes, lotions, milks, mousses, gels, oils, tonics, and sprays. Preferably the active(s), respectively the active blends are formulated into lotions, creams, gels, and tonics. These product forms may be used for several applications, including, but not limited to, hand and body lotions, facial moisturizers, anti-ageing preparations, make-ups including foundations, and the like. Any additional components required to formulate such products vary with product type and can be routinely chosen by one skilled in the art.

Suitable composition according to the invention are leave-on or rinse-off products, and include any product applied to the human body. Preferred in all embodiments of the present invention are leave-on products.

Rinse-off as well as leave-on as used herein is defined as per the Regulation (EC) No. 1223/2009 of the European Parliament and of the Council of 30 Nov. 2009 on cosmetic products (recast), i.e. a cosmetic product or composition is a rinse-off one when it is intended to be removed after application on skin, hair or mucous membranes of a human subject, whereas it is a leave-on one when it is intended to stay in prolonged contact with the skin, the hair or the mucous membranes.

If cosmetic or dermatological compositions of the present invention are formulated as an aerosol and applied to the skin as a spray-on product, a propellant is added to the cosmetic composition.

The cosmetic or dermatological compositions according to the present invention can be prepared by conventional methods in the art such as e.g. by admixing an active or an active blend according to the present with the cosmetically acceptable carrier.

The cosmetic or dermatological compositions of the invention (including the carrier) may comprise further conventional adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such cosmetic or dermatological compositions.

In accordance with the present invention, the cosmetic or dermatological compositions according to the invention may also comprise further cosmetically active ingredients conventionally used in cosmetic and/or dermatological compositions. Exemplary active ingredients encompass skin lightening agents; UV-filters, agents for the treatment of hyperpigmentation; agents for the prevention or reduction of inflammation; firming, moisturizing, soothing, and/or energizing agents as well as agents to improve elasticity and skin barrier.

Examples of cosmetic or dermatological excipients, diluents, adjuvants, additives as well as active ingredients commonly used in the skin care industry which are suitable for use in the cosmetic or dermatological compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of further active ingredients as well as the excipients, diluents, adjuvants, additives etc. can, based on the desired product form and application, easily be determined by the skilled person. The additional ingredients can either be added to the oily phase, the aqueous phase or separately as deemed appropriate.

The further cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The cosmetic or dermatological compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, alcoholic gel, lipogel, one-or multiphase solution or vesicular dispersion or other usual forms, which can also be applied by pens, as masks or as sprays.

If the cosmetic or dermatological composition is an emulsion, such as in particular an O/W, W/O, Si/W, W/Si, O/W/O, W/O/W multiple or a pickering emulsion, then the amount of the oily phase present in such cosmetic or dermatological emulsions is preferably at least 10 wt.-%, such as in the range of 10 to 60 wt.-%, preferably in the range of 15 to 50 wt.-%, most preferably in the range of 15 to 40 wt.-%, based on the total weight of the cosmetic or dermatological composition.

In one embodiment, the cosmetic or dermatological compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the cosmetic or dermatological composition according to the invention is an O/W emulsion, then it contains advantageously at least one O/W-or Si/W-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd.), diethanolamine cetyl phosphate (e.g. as Amphisol® DEA from DSM Nutritional Products Ltd.), potassium cetyl phosphate (e.g. as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt. %, in particular in the range of 0.5 to 6 wt.-%, such as more in particular in the range of 0.5 to 5 wt.-%, such as most in particular in the range of 1 to 4 wt.-%, based on the total weight of the cosmetic or dermatological composition.

Particular suitable O/W emulsifiers to be used in the cosmetic or dermatological compositions according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate.

A particular suitable O/W emulsifier to be used in the cosmetic or dermatological compositions according to the invention is potassium cetyl phosphate e.g. commercially available as Amphisol® K at DSM Nutritional Products Ltd Kaiseraugst.

Another particular suitable class of O/W emulsifiers are non-ionic self-emulsifying systems derived from olive oil e.g. known as (INCI Name) cetearyl olivate and sorbitan olivate (chemical composition: sorbitan ester and cetearyl ester of olive oil fatty acids) sold under the tradename OLIVEM 1000.

In one particular embodiment, the invention relates to cosmetic or dermatological compositions with all the definitions and preferences given herein in the form of O/W emulsions comprising an oily phase dispersed in an aqueous phase in the presence of an O/W emulsifier wherein the O/W emulsifier is potassium cetyl phosphate. The amount of oily phase in such O/W emulsions is preferably at least 10 wt.-%, more preferably in the range of 10 to 60 wt.-%, most preferably in the range of 15 to 50 wt.-%, such as in the range of 15 to 40 wt.-%.

The cosmetic or dermatological compositions according to the invention in general have a pH in the range of 3 to 10, preferably a pH in the range of 4 to 8 and most preferably a pH in the range of 4 to 7.5. The pH can easily be adjusted as desired with suitable acids, such as e.g. citric acid, or bases, such as sodium hydroxide (e.g. as aqueous solution), triethanolamine (TEA Care), Tromethamine (Trizma Base) and Aminomethyl Propanol (AMP-Ultra PC 2000), according to standard methods in the art.

The following examples are provided to further illustrate the effects of the present invention. These examples are illustrative only and are not intended to limit the scope of the invention in any way.

### EXAMPLES

The experiment was designed to investigate the skin and microbiome protection benefits of a sugar blend comprising psicose, mannose, fructose and glucose by assessing skin integrity by immunostaining of Cytokeratin-1 after S. aureus pathogenic colonization.

### Test Substances

The following substances were topically applied on the 3D skin tissue:
Vehicle control (ultrapure water)
Sugar premix (at 1% in ultrapure water (prepared by isomerization of plant derived glucose; (approx. 25% glucose, 15% fructose, 2% mannose, 2.5% psicose and 50% water)

### Methods

### 1. Preparation of 3D Skin

Primary adult human dermal fibroblasts were embedded into a fibrin matrix to produce dermal equivalents (DEs). The DEs were cultured to allow the fibroblasts to remodel the matrix. Primary neonatal human keratinocytes were applied to the DE surface and cultured under liquid for 48 hours. 3D skin was cultured at the air liquid interface until a stratified epidermis was formed. Incubation conditions for all cultures were 37° C. in 5% (v/v) CO² at >95% RH.

2. Test Procedure 3D skin was kept during the entire study in deep well plates. Maintenance medium was replaced the same day of colonization with commensal consortium.
2.1. Cutibacterium acnes NCTC 737 was grown for 5 days anaerobically at 37° C. using a Reinforced Clostridial Agar Medium.
2.2. Staphylococcus epidermidis NCTC 11047 and Corynebacterium striatum NCTC 764 were grown for 18-24 hours aerobically using Mueller-Hinton Agar and Aerobic Corynebacteria Agar, respectively.
2.3. Using inoculation buffer GS25, bacteria from the cultures mentioned above were used to prepare the initial inoculum mix containing ˜1.1×106 cfu mL−1 of each bacterium (3×Mix Consortium).
2.4. 10 μL of the initial inoculum were used to colonize each of the 3D skin units with ˜104 cfu cm⁻² of the bacterium.
2.5. 3D skin was incubated at 37° C. in 5% (v/v) CO2 at >95% RH for ˜2 hours (until 3D skin surface had dried).
2.6. Colonized 3D skin units were treated with either 10 μL of each test item or vehicle control.
2.7. 3D skin was incubated at 37° C. in 5% (v/v) CO² at >95% RH for ˜24 hours.
2.8. The same day 3D skin was colonized with the 3×Mix, Staphylococcus aureus strain NCTC 13435 was grown in Mueller-Hinton Agar aerobically at 37° C. for 18-24 hours.
2.9. Bacteria from the overnight culture of Staphylococcus aureus type strain NCTC 13435 mentioned above were used to prepare a bacterial suspension stock in buffer GS25.
2.10. 3D skin previously colonised with 1×104 cfu/cm² of the 3×Mix were infected with 102 cfu/cm−2 of S. aureus.
2.11. Colonised 3D skin was incubated at 37° C. in 5% (v/v) CO2 at >95% RH for ˜48 hours

Approximately 72 hours post-treatment with test items the tissue was fixed in 10% neutral buffered formalin, processed, paraffin embedded and sectioned. Immunohistochemistry and staining quantification was carried out to cytokeratin-1 expression in the tissue (3 test samples: untreated, vehicle control & sugar premix).

### Results (Histology): Cytokeratin Expression Versus Untreated

| | Vehicle control | Sugar premix |
|---|---|---|
| Cytokeratin-1 expression vs. untreated | 3% | 71% |
| Delta over vehicle control | — | +68% |

The results shows that 1% of the blend of sugars increased the tissue expression of Cytokeratine-1 in dermal fibroblasts colonized with C. acnes, S. Epidermidis, C. striatum and S. aureus by 68% compared to the expression obtained in samples treated with the vehicle control only.

1. Cosmetic use of a composition comprising an effective amount of a blend of sugars comprising psicose, mannose, fructose and glucose in a dermatological treatment as agent for increasing the expression of cytokeratin-1 in skin tissue, wherein the microbiome of the skin comprises one or more of C. acnes, S. Epidermidis, C. striatum and S. aureus or wherein the microbiome of said skin comprises one or more of C. acnes, S. Epidermidis and C. striatum and is exposed to or endangered to be exposed to S. aureus.

2. The cosmetic use according to claim 1, wherein the blend of sugars is used to strengthen skin's self-defense mechanisms and maintain skin integrity of said skin.

3. The cosmetic use according to claim 1, wherein the composition is applied onto the skin before exposure to S. aureus, followed by contacting the skin with S. aureus.

4. The cosmetic use according to claim 1, wherein the effective amount of psicose is selected in the range from 0.0001 to 0.5 wt.-%, preferably from 0.005 to 0.25 wt.-%, most preferably from 0.0075 to 0.2 wt.-%, based on the total weight of the composition.

5. The cosmetic use according to claim 1, wherein the effective amount of mannose is selected in the range from 0.001 to 0.5 wt.-%, preferably from 0.005 to 0.25 wt.-%, most preferably from 0.0075 to 0.2 wt.-%, based on the total weight of the composition.

6. The cosmetic use according to claim 1, wherein the effective amount of fructose is selected in the range from 0.01 to 2 wt.-%, preferably from 0.05 to 1 wt.-%, most preferably from 0.05 to 0.75 wt.-%, based on the total weight of the composition.

7. The cosmetic use according to claim 1, wherein the effective amount of glucose is selected in the range from 0.01 to 3 wt.-%, preferably from 0.05 to 2.5 wt.-%, most preferably from 0.075 to 2 wt.-%, based on the total weight of the composition.

8. The cosmetic use according to claim 1, wherein the sugars are incorporated into the composition in the form of an aqueous premix comprising, based on the total weight of the premix
a) from 1 to 5 wt.-%, preferably from 2 to 3 wt.-% of psicose,
b) from 1 to 5 wt.-%, preferably from 1.5 to 3 wt.-% of mannose,
c) from 10 to 30 wt.-%, preferably from 10 to 20 wt.-% of fructose, and
d) from 15 to 30 wt.-%, preferably from 20 to 30 wt.-% of glucose.

9. The cosmetic use according to claim 8, wherein the premix is obtained by isomerization of plant derived glucose.

10. A method to prevent or treat skin conditions caused by a reduced expression of cytokine-1 in skin, wherein the microbiome of said skin comprises one or more of C. acnes, S. Epidermidis and C. striatum and wherein the skin is to be exposed to S. aureus, said method comprising the following steps:
a) providing a topical composition comprising a blend of sugars comprising psicose, mannose, fructose and glucose, and
b) applying an amount of said topical composition to human skin or scalp that is sufficient to increase cytokeratin-1 expression, followed by
c) exposure of said skin to S. aureus.

11. A method of treating a skin condition in which a reduced cytokeratin-1 expression contributes to the pathology, and/or symptoms, and/or progression of said skin condition and wherein the microbiome of said skin comprises one or more of C. acnes, S. Epidermidis, C. striatum and S. aureus or wherein the microbiome of said skin comprises one or more of C. acnes, S. Epidermidis and C. striatum and is exposed to or endangered to be exposed to S. aureus, said method comprising administering an effective amount of a blend of sugars comprising psicose, mannose, fructose and glucose to a subject in need thereof.

12. A method to strengthen skin's self-defense mechanism and maintain skin integrity in skin wherein the microbiome of said skin comprises one or more of C. acnes, S. Epidermidis, C. striatum and S. aureus or wherein the microbiome of said skin comprises one or more of C. acnes, S. Epidermidis and C. striatum and is exposed to or endangered to be exposed to S. aureus, said method comprising the step of applying to a skin area in need of said strengthening an effective amount of a blend of sugars comprising psicose, mannose, fructose and glucose.

13. Cosmetic use of a blend of sugars comprising psicose, mannose, fructose and glucose as cytokeratin-1 promotor in skin wherein the microbiome of said skin comprises one or more of C. acnes, S. Epidermidis, C. striatum and S. aureus or wherein the microbiome of said skin comprises one or more of C. acnes, S. Epidermidis and C. striatum and is exposed to or endangered to be exposed to S. aureus.
